(19) United States
(12) Patent Application Publication
(10) Pub. No.: US 2010/0041146 A1
(43) Pub. Date: Feb. 18, 2010

(54) THREE-DIMENSIONAL CELL CULTURE CARRIER AND METHOD FOR CELL CULTURE USING THE SAME

(75) Inventors: Hiroshi Kambayashi, Tokyo (JP); Masanobu Tsuda, Tokyo (JP); Noriaki Sato, Tokyo (JP); Keizaburo Miki, Kanagawa (JP); Nanako Mitani, Kanagawa (JP)

Correspondence Address: HAMRE, SCHUMANN, MUELLER & LARSON, P.C., P.O. BOX 2902, MINNEAPOLIS, MN, 55402-0902, US

(73) Assignee: NIPPON SHEET GLASS COMPANY, LIMITED, Tokyo (JP)

(21) Appl. No.: 12/523,003

(22) PCT Filed: Jan. 11, 2008
(86) PCT No.: PCT/JP2008/050286

§ 371 Date: Aug. 28, 2009

(30) Foreign Application Priority Data
Jan. 12, 2007 (JP) 2007-004691

(51) Int. Cl. C12N 5/02; C12M 1/00

(52) U.S. Cl. 435/383; 435/283.1

(57) ABSTRACT

A three-dimensional cell culture carrier of the present invention includes a fibrous structure having a three-dimensional space for cell culture. The fibrous structure is formed of a plurality of intertangled fibers. Each of the fibers used in the fibrous structure is made of a material having a visible light transmittance of 40% or more (preferably 50% or more) when it is formed into a shape having a thickness of 3 mm. Examples of such fibers include glass fibers. The fiber has an aspect ratio of 1 or more, and a fiber diameter of 100 μm to 700 μm.

### TECHNICAL FIELD

The present invention relates to a three-dimensional cell culture carrier useful for three-dimensional culture of cells, and a method for cell culture using the same.

### BACKGROUND ART

There has been suggested a plane cell culture carrier made of a calcium phosphate-based compound as a cell culture carrier that allows the state of cells in culture (the appearance of attachment or growth of cells) to be observed with an optical microscope (see JP 2004-173502 A). In the plane cell culture carrier, cells are attached to and grown on one surface of the carrier, while the state of cells can be observed from the opposite surface (the other surface).

Further, a cell culture carrier produced by entangling carbon fibers in a three-dimensional space is suggested that enables multilayered cells or cells with a high density to be grown (see JP 2004-135668 A). Furthermore, there also has been disclosed a cell culture carrier where inorganic porous materials such as porous glass is used, thereby allowing cells to be cultured efficiently and observed easily (see JP 2006-141290 A).

JP 2003-509021 T further discloses a cell growth substrate including a water-soluble glass matrix. The cell growth substrate is formed of glass fibers each having a fiber diameter ranging from 20 to 30 μm, or glass particles having an average diameter ranging from 15 μm to 6 mm, for example.

In the above-described conventional plane cell culture carriers, however, the transmittance with respect to the light with a wavelength of 600 nm is as low as about several percent when the average thickness is 1 mm. Accordingly, there are some cases where the state of cells is difficult to observe. Moreover, since the cell culture carrier is in a plane, there has been a difficulty in establishing a three-dimensional solid culture that replicates in vivo condition. There has been a problem that simple increase in the thickness for the purpose of a three-dimensional cell culture causes the reduction in the visible light transmittance, so as to prevent observation of cells. As a result, the cells are required to be separated from the carrier in order to observe the cells.

Further, although the cell culture carrier formed by using carbon fibers allows a three-dimensional solid culture of cells, there has been a difficulty in detailed observation of cells in the process of culture with an optical microscope, because the visible light transmittance of a carbon fiber in itself is low. Furthermore, in the case of using porous glass for a cell culture carrier, it is sometimes difficult to obtain a sufficient light transparency, depending on its shape. It is thus difficult to achieve both cell culture with an improved efficiency and easy observation of cells.

The above-described cell growth substrate produced by using water-soluble glass has a visible light transmittance to an extent where observation of cells is possible. However, when glass fibers each having a fiber diameter of 20 to 30 μm or glass particles having an average diameter of 15 μm to 6 mm are used, it is difficult to ensure a three-dimensional space with an environment similar to the in vivo environment, and difficult to obtain the morphology and function of cells to be cultured in the same manner as those of cells in vivo.

### DISCLOSURE OF INVENTION

The present invention has been accomplished in view of these conventional problems and it is therefore an object of the present invention to provide a three-dimensional cell culture carrier and a method for cell culture that allow easy observation of cells in the process of culture in detail with an optical microscope or the like, and cell culture in a similar condition to that of cell growth in vivo.

A three-dimensional cell culture carrier according to the present invention includes a fibrous structure provided with a three-dimensional space for cell culture. The fibrous structure is formed of a plurality of interconnected fibers. Each fiber is made of a material with a visible light transmittance of 40% or more when it is formed into a shape having a thickness of 3 mm, and has an aspect ratio of 1 or more and a fiber diameter ranging from 100 μm to 700 μm. It should be noted that visible light herein means light with a wavelength of 380 nm to 780 nm. Further, the aspect ratio is a value calculated from the fiber length divided by the fiber diameter.

The cells in the process of culture can be observed in detail with an optical microscope or the like, because a fibrous structure employed in the three-dimensional cell culture carrier of the present invention is formed by fibers made of a material with a high visible light transmittance. Further, in the three-dimensional cell culture carrier of the present invention, a plurality of fibers are interconnected to form a fibrous structure, so that a variety of shape, size and porosity or the like can be achieved easily. Furthermore, the fiber has an aspect ratio of 1 or more, and its fiber diameter is from 100 μm to 700 μm, so that a fibrous structure having pores with a size suitable for cell culture can be produced easily. Accordingly, a three-dimensional space with an environment similar to the in vivo environment is feasible, thereby allowing the morphology and function of the cultured cells to occur in the same manner as those of cells in vivo. Moreover, a fibrous structure with a high visible light transparency is feasible, while preventing scattered light sufficiently, by selecting the fiber diameter and the fiber length appropriately in accordance with the intended shape (e.g. thickness) due to use of fibers for its formation. That is, according to the present invention, a three-dimensional cell culture carrier with a high visible light transparency can be achieved, without being restricted by the shape. Further, according to the three-dimensional cell culture carrier of the present invention, it is also advantageous that the cultured cells are easier to remove, compared to conventional three-dimensional cell culture carriers. In other words, according to the three-dimensional cell culture carrier of the present invention, the cells can be removed from the three-dimensional cell culture carrier as easily as from a two-dimensional culture container such as a Petri dish.

As described above, the three-dimensional cell culture carrier of the present invention allows detailed observation with an optical microscope or the like and cell culture under a condition similar to that of cell growth in vivo to be achieved concurrently.

A method for cell culture according to the present invention includes the steps of providing a cell culture carrier with cells, and supplying a broth to the cell culture carrier thereby allowing the cells to grow. The cell culture carrier used therein is the above-described three-dimensional cell culture carrier of the present invention. Therefore, the method for cell culture according to the present invention enables cells to be cultured efficiently under a condition similar to that of cell growth in vivo, and to be observed with an optical microscope.

### BRIEF DESCRIPTION OF DRAWINGS

FIG. 1A is an explanatory perspective view illustrating a step in an example of the cell culture method of the present invention.

FIG. 1B is an explanatory perspective view illustrating a step in an example of the cell culture method of the present invention.

FIG. 1C is an explanatory perspective view illustrating a step in an example of the cell culture method of the present invention.

FIG. 2 is an image showing the appearance of cell culture observed with an optical microscope in the three-dimensional cell culture carrier of an example (sample 2-5) of the present invention.

FIG. 3 is an image showing the appearance of cell culture observed with an optical microscope in the three-dimensional cell culture carrier of an example (sample 2-6) of the present invention.

### BEST MODE FOR CARRYING OUT THE INVENTION

Hereinafter, embodiments of the present invention are described.

<Three-Dimensional Cell Culture Carrier>

An embodiment of the three-dimensional cell culture carrier according to the present invention is described.

The three-dimensional cell culture carrier of this embodiment is formed by using a fibrous structure. Note that, although a three-dimensional cell culture carrier including only a fibrous structure formed of a plurality of fibers is described as an example in this embodiment, the three-dimensional cell culture carrier of the present invention may include, for example, an adhesive agent in order to interconnect the plurality of fibers that constitute the fibrous structure. Further, a protein, a peptide, an amino acid, a chemical compound or the like that promote adhesion, growth and differentiation of cells may be coated or bonded in order to add more properties to the fibrous structure. Examples include, but are not restricted to, collagen, fibronectin, laminin, polylysine, poly ornithine, polyethyleneimine, antibodies, ligands, receptor proteins, and adhesion factors.

The fibrous structure has a solid structure including a three-dimensional space (pores), and is formed of a plurality of interconnected fibers. Examples of the fibers used in the fibrous structure of this embodiment include glass fibers or synthetic fibers. A detailed description thereof will follow later. In the case of using glass fibers, sintered glass fibers may be used for a fibrous structure. In order to ensure a three-dimensional space suitable for cell growth, the porosity of the fibrous structure is preferably from 30% to 70%, more preferably from 38% to 55%. It should be noted that the porosity of a fibrous structure herein means a percentage of the volume occupied by the fibrous structure excluding the volume of fibers therefrom, with respect to the volume occupied by the fibrous structure. That is, the porosity is a value calculated from the following formula:

Porosity(%)=(V_{R}−V_{G})×100/V_{R}

wherein V_{R}: volume of fibrous structure, and
V_{G}: volume of fibers.

Further, in the present invention, volume V_{R} (the volume of fibrous structure) occupied by the fibrous structure can be calculated from: radius33 radius×n×thickness, for example, when the fibrous structure is coin-like shape, and the volume of the fibers can be calculated from the density and mass of the fibers.

The fibers used for the fibrous structure are required to be made of materials with a high visible light transmittance in order to achieve a three-dimensional cell culture carrier that is transparent enough so that the cells in culture can be observed. In view of this, the fibers are made of a material having a visible light transmittance of 40% or more, when they are formed into a shape having a thickness of 3 mm. It is preferable to use fibers made of a material having a visible light transmittance of 50% or more (more preferably 80% or more), when the fibers are formed into a shape having a thickness of 3 mm (more preferably, 5 mm). Examples of fibers made of such a material with a sufficient visible light transmittance include glass fibers or synthetic fibers (for example, fibers made of acrylic, polyester, rayon, nylon, polystyrene, and the like). Above all, glass fibers preferably are used. When the fibrous structure is formed of glass fibers, a three-dimensional cell culture carrier that is transparent with respect to visible light can be achieved, as well as the surface condition thereof is more smooth and chemically stable, so that the cells can be attached thereto easily. Furthermore, glass fibers are unidirectionally continuous. Such unidirectional continuity of glass fibers may be preferable for cell culture.

There are several kinds of glass fibers corresponding to the difference in the content ratio of composition components. It is desirable to select one of them appropriately, taking into consideration, for example, the creation of an environment suitable for cell growth. Since cell culture is carried out generally with a slightly alkali, about pH7.4, glass fibers with C-glass composition that have an appropriate water resistance in this environment are preferably used. Also, glass fibers having E-glass composition or A-glass composition are available.

The fiber used for the three-dimensional cell culture carrier of the present invention has an aspect ratio (given by its fiber length divided by its fiber diameter) of 1 or more (preferably 1 to 10). Use of such fibers allows a fibrous structure having appropriate pores to be produced, so that a three-dimensional cell culture carrier more suitable for cell culture can be obtained. In order to obtain a fibrous structure having pores with a size that facilitates easier cell culture, it is desirable to use fibers each with an aspect ratio of 1.5 or more.

A plurality of fibers constituting the fibrous structure each have a fiber diameter ranging from 100 μm to 700 μm, preferably 250 μm to 500 μm (e.g. 300 μm). Use of fibers having a fiber diameter of 100 μm or more allows a fibrous structure having pores with a size suitable for cell culture to be produced easily while reducing scattered light, so that the cells can be observed easily. If the fiber diameter is less than 100 μm, the curvature of fibers in the form of a fibrous structure becomes large, thereby causing a difficulty in attaching cells to the fibers. Further, if the fiber diameter is less than 100 μm, an appropriate porosity is difficult to ensure, resulting in lack of space for cell culture. If the fiber diameter exceeds 700 μm, the pores in a fibrous structure become too large, and thereby stable and three-dimensional accumulation of cells becomes difficult. Accordingly, fibers with the fiber diameter of 700 μm or less should be used.

A plurality of fibers constituting a fibrous structure have a fiber length of preferably 500 μm to 50000 μm, more preferably 500 μm to 6000 μm, and further preferably 500 μm to 3000 μm. In other words, it is preferable that the fiber length distribution of the plurality of fibers (fiber group) constituting a fibrous structure be included in the range of 500 μm to 50000 μm, more preferably in the range of 500 μm to 6000 μm, and further preferably in the range of 500 μm to 3000 μm. Use of a fiber group having such a fiber length distribution allows a fibrous structure having pores suitable for cell culture to be produced easily, and thus a three-dimensional cell culture carrier suitable for cell culture can be obtained.

For example, when glass fibers are used in the present invention, glass fibers are produced by melting and spinning glass having a particular composition so as to have a particular fiber diameter. Then, the produced glass fibers are roughly cut, crushed and classified, thereby achieving the above-described fiber diameter, fiber length and aspect ratio. Thus produced glass fiber has a substantially cylindrical shape, and the surface thereof is smooth.

The shape of the three-dimensional cell culture carrier of the present invention is not specifically limited. It can be determined appropriately in accordance with the shape of an incubator that accommodates the carriers. For example, when using a microplate for cell culture provided with a plurality of wells (concave portions), each serving as an incubator, the shape of the three-dimensional cell culture carrier may be a coin-like shape capable of being accommodated in the well. By changing the size (outer diameter) of the coin-like shape, the carrier can be accommodated in a well of any size (e.g. a well of each size where the number of the wells arranged on a single plate is 12, 24, 48 or 96).

The three-dimensional cell culture carrier of the present invention may be a hollow cylindrical shape, incorporated into a radial flow type culture device (e.g. a radial flow type reactor manufactured by ABLE Corporation). In this case, a three-dimensional cell culture carrier having a hollow cylindrical shape can be achieved by filling the inside of the reactor of the culture device with a fibrous structure. The three-dimensional cell culture carrier of the present invention may have such a shape that the cultured cells are easy to remove. The shape is feasible, for example, by rendering the fibrous structure divisible into a plurality of sections. Particularly, when the carrier is incorporated into a radial flow type culture device, the fibrous structure may be formed by laminating a plurality of annular sheets each formed of fibers.

As described above, the three-dimensional cell culture carrier according to the present invention is formed by using a fibrous structure. Therefore, for application, it may be formed into a particular shape, or filled into a device to be used. Accordingly, a container and filling operation are not always required unlike the cell culture carriers made of conventional porous glass beads (e.g. “Siran” manufactured by Schott Glaswerke, Germany), and handling is easy. Further, the unevenness in the filling rate is reduced, and thereby the unevenness in the cultured cells also is reduced.

Hereinafter, an example of a method for producing a three-dimensional cell culture carrier in accordance with the present invention is described. It should be noted that although a method for producing a three-dimensional cell culture carrier with a coin-like shape by using glass fibers is described as an example, the three-dimensional cell culture carrier of the present invention is not limited to this.

Glass fibers are produced to have a particular fiber diameter by melting and spinning glass having a particular composition. After being roughly cut and crushed, the produced glass fibers are classified into those having a particular fiber length distribution. The method of classification may be, but not restricted to, dry sieving by means of a test sieve as specified in JIS (JIS Z 8801). A plurality of glass fibers thus obtained having a particular fiber diameter and fiber length are packed into a ceramic tube having a particular internal diameter and length. Maintaining this condition, calcination is carried out at a temperature of, for example, 650° C. to 850° C. for 1 to 3 hours, followed by cooling. After cooling, the glass fibers removed from the furnace are sliced over the ceramic tube so as to have a particular thickness, and sintered glass fibers (fibrous structure) are removed from the ceramic tube. Thus, a three-dimensional cell culture carrier having a coin-like shape can be produced. As already mentioned, examples of the glass composition of the glass fibers include C-glass composition, E-glass composition and A-glass composition. Examples of the compositions are as indicated in Table 1 below. The composition rates indicated in Table 1 are expressed as mass %.

TABLE 1

| | C-GLASS | E-GLASS | A-GLASS |
|---|---|---|---|
| | COMPOSITION | COMPOSITION | COMPOSITION |
| SiO₂ | 65 to 72 | 52 to 56 | 70 to 73 |
| Al₂O₃ | 1 to 7 | 12 to 16 | 0 to 5 |
| CaO | 4 to 11 | 16 to 25 | 5 to 10 |
| MgO | 0 to 5 | 0 to 6 | 0 to 5 |
| B₂O₃ | 0 to 8 | 5 to 13 | — |
| Na₂O + K₂O | 9 to 17 | 0 to 0.8 | 10 to 18 |
| ZnO | 0 to 6 | — | — |

<Cell Culture Method>

In the cell culture method of the present invention, broth is fed to the above-described cell culture carrier of the present invention, thereby allowing cells to grow.

Examples of cells to be used in the cell culture method of the present invention include, but are not restricted to, cells derived from tissue and organ, such as fibroblasts, chondrocytes, (mesenchymal, haematopoietic and embryonic) stem cells, nerve cells, epithelial cells, osteoblasts, endothelial cells, cardiomyocytes, myoblasts, pancreatic cells, hepatocytes, and further, tumorigenic cells derived from animal, cells derived from animal, insect and plant including cell lines, and the like. Cells produced by transgenesis in these cells also are included. Note that “cells” herein include a cell-free system having protein synthetic capacity or DNA synthetic capacity.

The cell culture method of the present invention may employ, for example, a microplate for cell culture having a plurality of wells, or a radial flow type bioreactor accommodating a three-dimensional cell culture carrier. When employing a radial flow type bioreactor, it is possible to mass produce cells, and also to extract a large amount of metabolites from the cultured cells.

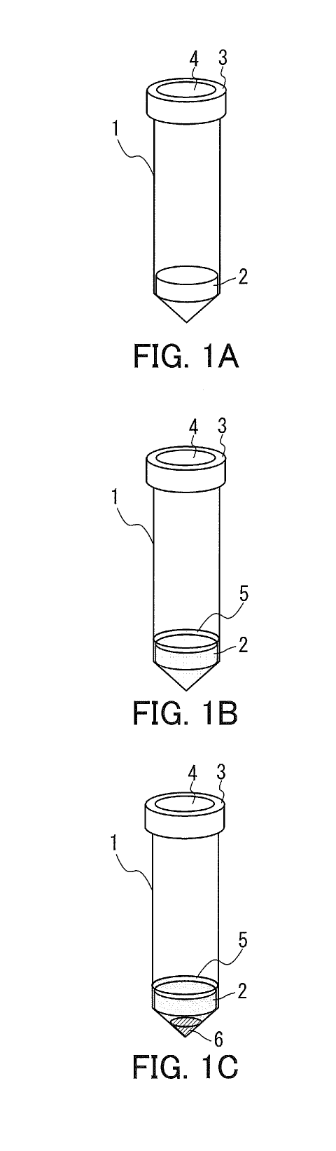

As another example of the cell culture method of the present invention, an example employing a conical centrifugation tube is described. As described above, the three-dimensional cell culture carrier of the present invention can change its shape appropriately, corresponding to the shape of the incubator that accommodates the carrier. Accordingly, cells can be cultured in a conical centrifugation tube by changing the shape of the three-dimensional cell culture carrier of the present invention into a shape suitable for accommodation in a conical centrifugation tube. With reference to FIGS. 1A to 1C, the method for cell culture in a conical centrifugation tube is described below.

First, as shown in FIG. 1A, a three-dimensional cell culture carrier 2 of the present invention is arranged inside a conical centrifugation tube 1. The three-dimensional cell culture carrier 2 is coin-like shape, and formed into a size suitable for accommodation inside the conical tube 1. An example employing a cap 3 with a HEPA filter 4 is described here. Next, cells are disseminated to the three-dimensional cell culture carrier 2 inside the conical tube 1, thereby allowing cells to be cultured in broth 5 (see FIG. 1B). After the completion of culture, an enzyme (e.g. trypsin) is introduced into the conical tube 1, and the enzyme causes the cells to separate from the three-dimensional cell culture carrier 2. Subsequently, centrifugation is carried out, and precipitate 6 of cells is collected at the bottom of the conical tube 1 (see FIG. 1C). Finally, the three-dimensional cell culture carrier 2 and the supernatant are removed to retrieve the precipitate 6 of the cells, and thus the cultured cells are obtained.

Conventionally, cells are cultured in another container, and after the completion of culture, the cultured cells are transferred into a conical centrifugation tube to be centrifuged. Because of the necessity for this operation, there has been a problem of bacterial contamination or misidentification of cells that could happen during the transferring operation. In contrast, in the method described above with reference to FIGS. 1A to 1C, there are no problems of bacterial contamination and the like, because operations of cell culture and detachment of the cultured cells can be carried out in a single container.

The broth to be used in the cell culture method of the present invention is not specifically limited; it may be selected appropriately, corresponding to cells to be cultured.

According to the cell culture method of the present invention, a three-dimensional cell culture is feasible, so that cells can be cultured in a condition similar to the in vivo condition. In addition, cells in the process of culture can be observed easily, because the three-dimensional cell culture carrier to be used is transparent with respect to visible light.

### EXAMPLES

Hereinafter, the present invention is described more specifically by way of examples.

### Example 1

### Method for Producing Three-Dimensional Cell Culture Carrier

Glass fibers with a fiber diameter of 300 μm were produced by melting and spinning glass with the C-glass composition indicated in Table 2, and the produced glass fibers were roughly cut and crushed. After that, the glass fibers were classified into those having a fiber length distribution of 500 to 1500 μm. Specifically, they were classified by means of test sieves specified in JIS, by dry sieving. The glass fibers that passed through a sieve with a mesh size of 710 μm (pre-sieve) and remained on a sieve with a mesh size 300 μm (post-sieve) were used as sintered body material. 3 g of thus obtained glass fibers (sintered body material) were packed into a ceramic tube with an internal diameter of 13 mm and length of 50 mm. Maintaining the condition, calcination was carried out at a temperature of 670° C. for 1 hour, followed by furnace cooling. After cooling, the glass fibers taken out from the furnace were sliced over the ceramic tube with a diamond cutter into 2 to 3 mm thickness, and sintered glass fibers (fibrous structure) were taken out from the inside of the ceramic tube. Thus, a three-dimensional cell culture carrier having a coin-like shape with a thickness of 2 to 3 mm and a diameter of 12 to 13 mm was produced. The glass with C-glass composition used in this example had a visible light transmittance of 80% or more, when it was formed into a shape having a thickness of 3 mm (specifically, it had a transmittance of 95% with respect to light having a wavelength of 600 nm). In this regard, the visible light transmittance in this example was measured by means of spectrophotometer with respect to the glass with C-glass composition used in this example that had been formed into 3 mm-thick glass. This is the same in the following examples. The porosity of the three-dimensional cell culture carrier in this example was 47%.

TABLE 2

| | COMPOSITION |
|---|---|
| | (mass %) |
| SiO₂ | 68.7 |
| Al₂O₃ | 2.5 |
| CaO | 6.5 |
| MgO | 2.5 |
| B₂O₃ | 4.4 |
| Na₂O + K₂O | 15.4 |
| ZnO | 0 |

### [Cell Culture Method]

The thus produced three-dimensional cell culture carrier (sterilized) was accommodated in each well of a 24-well plate (manufactured by Becton, Dickinson and Company, “353047”) in a safety cabinet (manufactured by SANYO Electric Co., Ltd., “MHE-130AB3”) aseptically, and hepatoma cells were cultured by the following procedures.

(1) The cell suspension used in this example was obtained by suspending hepatoma cells cultured in a 10 cm-tissue culture dish (manufactured by Becton, Dickinson and Company, “353003”) into a culture medium. Hepatoma cells were cultured at 37° C. under a mixed gas of 95 vol % air and 5 vol % carbon dioxide (CO₂), until 80% to 90% confluent (culture surface saturation). After the removal of the culture medium, the cells were cleaned twice with PBS (−). The cells were separated by using 2 mL of 0.25% Trypsin-EDTA (manufactured by GIBCO, “25200-072”), and suspended into 8 mL of DMEM/F12 culture medium (manufactured by Sigma-Aldrich Corporation, “D8900”) with an added FBS (Fetal Bovine Serum) (manufactured by Bio West, “S1820”) to have a final concentration of 10%. The cell suspension was centrifuged with a cooling centrifugal machine (manufactured by TOMY CORPORATION, “EX-126”) (at 4° C. and 1000 rpm, for 2 minutes), followed by the removal of the supernatant. A culture medium (DMEM/F12 culture medium, “D8900”, manufactured by Sigma-Aldrich Corporation) was added thereto, and the cell suspension was diluted so that the number of cells was 2.0×10⁶ cells/mL. The prepared cell suspension was disseminated as 100 mL/well (2.0×10⁵ cells/well).

(2) The cell suspension and the carriers in the plate were put into a CO₂ incubator (manufactured by SANYO Electric Co., Ltd., “MCO-17A1C”), and incubated at 37° C. under a mixed gas of 95 vol % air and 5 vol % carbon dioxide (CO₂) for 1 hour.

(3) Another 900 μL of the culture medium of the same cell suspension as that used in (1) was added thereto (total dissemination amount: 2.0×10⁵ cells/mL/well).

(4) Cell culture was started.

(5) 24 hours later, the three-dimensional cell culture carriers were moved to a new well.

(6) The cells attached to the bottom of the well and the cells attached to the three-dimensional cell culture carrier were partitioned.

(7) After that, the culture medium (broth) was replaced repeatedly every 48 hours, and cell culture was continued eventually for 168 hours.

As a result, the eventual number of the cells attached to the three-dimensional cell culture carrier of this example was about 5×10⁵ cells. This was about 10 times the number of the cells that were cultured in a normal (two-dimensional) 24-well plate (about 5.12×10⁴ cells per 2 cm²).

In view of this result, it was confirmed that three-dimensional culture by using the three-dimensional cell culture carrier of the present invention leads to creation of a condition closer to the in vivo condition. Hemocytometer (manufactured by ERMA INC., “Neubauer line Haemocyto meter”) was used for counting the cell number per 1 mm². Since the fluid volume per 1 mm² was 0.1 mm³, the count result was multiplied by 10⁴ so that the cell number per 1 mL was calculated.

The cultured cells were observed with an optical microscope and the appearance of cell growth was clear.

### Example 2

Samples 2-1 to 2-11 of the three-dimensional cell culture carrier were produced in the same manner as Example 1, except that the fiber diameter and fiber length were changed. Classification was carried out by means of the combinations of sieves (pre-sieve and post-sieve) indicated in Table 3. The glass fibers that passed through the pre-sieve and remained on the post-sieve were used as sintered body material for each sample. The fiber diameters and fiber length distributions of the obtained glass fibers are indicated in Table 4.

TABLE 3

| | MESH SIZE OF | MESH SIZE OF |
|---|---|---|
| | PRE-SIEVE | POST-SIEVE |
| SAMPLE No. | (μm) | (μm) |
| 2-1 | 150 | 75 |
| 2-2 | 300 | 150 |
| 2-3 | 710 | 300 |
| 2-4 | 710 | 300 |
| 2-5 | 1000 | 710 |
| 2-6 | 1400 | 1000 |
| 2-7 | 710 | 300 |
| 2-8 | 1000 | 710 |
| 2-9 | 1400 | 1000 |
| 2-10 | 1000 | 710 |
| 2-11 | 1400 | 1000 |

Cells were cultured in the same manner as Example 1 by using these samples. Note that Sample 2-4 is the same as the three-dimensional cell culture carrier of Example 1. The eventually obtained cell numbers of these samples were compared to the cell number in the case of using the cell culture carrier manufactured by PENTAX CORPORATION (CELLYARD™HA, scaffold, φ13 mm×2 mm (no product number)) to be evaluated. The evaluation results are indicated in Table 3. The definitions of A, B, C and D in the evaluation results are as follows:

A: The cultured cell number is over 2.0 times that in the cell culture carrier manufactured by PENTAX CORPORATION;

B: The cultured cell number is over 1.5 times and no more than 2.0 times that in the cell culture carrier manufactured by PENTAX CORPORATION;

C: The cultured cell number is over 1.0 times and no more than 1.5 times that in the cell culture carrier manufactured by PENTAX CORPORATION; and

D: The cultured cell number is equal to or less than that in the cell culture carrier manufactured by PENTAX CORPORATION.

TABLE 4

| | FIBER | FIBER LENGTH | | EVALUA- |
|---|---|---|---|---|
| SAMPLE | DIAMETER | DISTRIBUTION | POROSITY | TION |
| No. | (μm) | (μm) | (%) | RESULT |
| 2-1 | 100 | 100 to 1000 | 29 | D |
| 2-2 | 100 | 150 to 5000 | 32 | C |
| 2-3 | 100 | 300 to 20000 | 48 | C |
| 2-4 | 300 | 500 to 1500 | 47 | A |
| 2-5 | 300 | 1000 to 3000 | 53 | A |
| 2-6 | 300 | 2000 to 6000 | 66 | B |
| 2-7 | 450 | 5000 to 25000 | 51 | B |
| 2-8 | 450 | 18000 to 42000 | 51 | B |
| 2-9 | 450 | 25000 to 50000 | 54 | B |
| 2-10 | 520 | 14000 to 38000 | 49 | B |
| 2-11 | 520 | 24000 to 48000 | 51 | B |

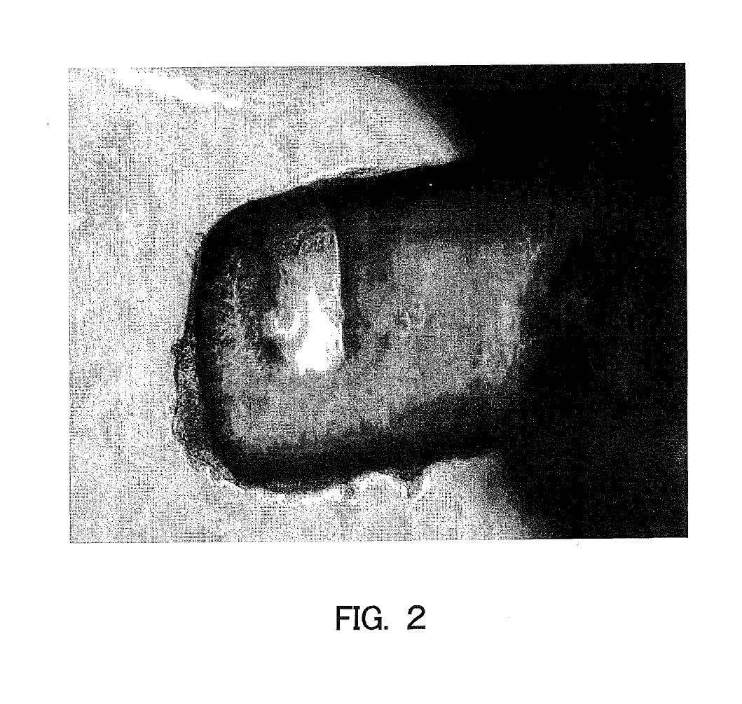

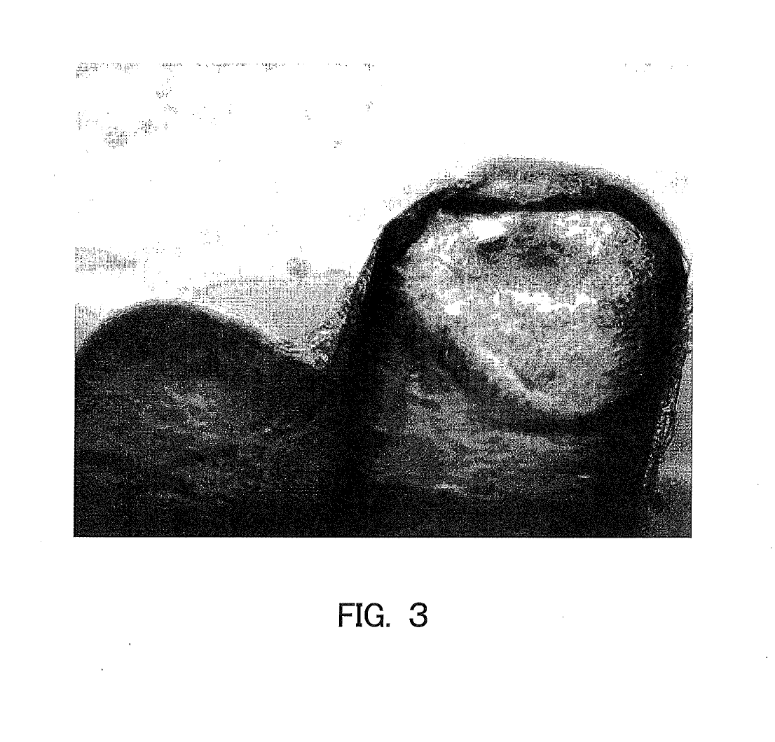

In view of the above results, it has been confirmed that the numbers of the cultured cells of Samples 2-2 to 2-11 with a porosity of 30% or more, and an aspect ratio of the fiber of 1.5 or more were more than the number of the cultured cells of the conventional cell culture carrier (the cell culture carrier manufactured by PENTAX CORPORATION). In addition, the cells could be observed in detail by means of an optical microscope with respect to all Samples 2-1 to 2-11. FIG. 2 is an optical micrograph showing an appearance where cells were cultured by using Sample 2-5, and FIG. 3 is an optical micrograph showing an appearance where cells were cultured by using Sample 2-6. It can be seen from these micrographs that, according to the three-dimensional cell culture carrier of the present invention, the appearance of cell growth can be observed sufficiently with an optical microscope.

### Example 3

Samples 3-1 to 3-4 of the three-dimensional cell culture carrier were produced in the same manner as Example 1 and cells were cultured in the same manner as Example 1, except that the glass fibers with E-glass composition indicated in Table 5 were used and the fiber diameters and fiber lengths were further changed. The evaluation results with respect to these samples that were obtained in the same manner as Example 2 are indicated in Table 6. The glass with E-glass composition used in this example had a visible light transmittance of 50% or more, when the thickness was 3 mm (for example, it had a transmittance of 60% with respect to light having a wavelength of 600 nm).

TABLE 5

| | COMPOSITION |
|---|---|
| | (mass %) |
| SiO₂ | 55.0 |
| Al₂O₃ | 14.3 |
| CaO | 23.0 |
| MgO | 0.2 |
| B₂O₃ | 5.8 |
| Na₂O + K₂O | 0.65 |
| ZnO | — |

TABLE 6

| | FIBER | FIBER LENGTH | | EVALUA- |
|---|---|---|---|---|
| SAMPLE | DIAMETER | DISTRIBUTION | POROSITY | TION |
| No. | (μm) | (μm) | (%) | RESULT |
| 3-1 | 300 | 500 to 1500 | 46 | C |
| 3-2 | 300 | 1000 to 3000 | 64 | C |
| 3-3 | 450 | 5000 to 25000 | 39 | A |
| 3-4 | 450 | 18000 to 42000 | 52 | C |

It has been confirmed that the numbers of the cultured cells of all Samples 3-1 to 3-4 were more than the numbers of the cultured cells of the conventional cell culture carrier (the cell culture carrier manufactured by PENTAX CORPORATION). In addition, the cells could be observed in detail with an optical microscope with respect to all Samples 3-1 to 3-4.

### Example 4

Samples 4-1 to 4-4 of the three-dimensional cell culture carrier were produced in the same manner as Example 1 and cells were cultured in the same manner as Example 1, except that the glass fibers with A-glass composition indicated in Table 7 were used and the fiber diameters and fiber lengths were changed further. The evaluation results with respect to these samples that were obtained in the same manner as Example 2 are indicated in Table 8. The glass with A-glass composition used in this example had a visible light transmittance of 60% or more, when the thickness was 3 mm (for example, it had a transmittance of 75% with respect to light having a wavelength of 600 nm).

TABLE 7

| | COMPOSITION |
|---|---|
| | (mass %) |
| SiO₂ | 72.8 |
| Al₂O₃ | 3.1 |
| CaO | 5.7 |
| MgO | 1.4 |
| B₂O₃ | — |
| Na₂O + K₂O | 17.0 |
| ZnO | — |

TABLE 8

| | FIBER | FIBER LENGTH | | EVALUA- |
|---|---|---|---|---|
| SAMPLE | DIAMETER | DISTRIBUTION | POROSITY | TION |
| No. | (μm) | (μm) | (%) | RESULT |
| 4-1 | 300 | 500 to 1500 | 39 | C |
| 4-2 | 300 | 1000 to 3000 | 52 | B |
| 4-3 | 450 | 5000 to 25000 | 38 | B |
| 4-4 | 450 | 18000 to 42000 | 45 | C |

It has been confirmed that the numbers of the cultured cells of all Samples 4-1 to 4-4 were more than the number of the cultured cells of the conventional cell culture carrier (the cell culture carrier manufactured by PENTAX CORPORATION). In addition, the cells could be observed in detail with an optical microscope with respect to all Samples 4-1 to 4-4.

### INDUSTRIAL APPLICABILITY

According to the three-dimensional cell culture carrier and cell culture method of the present invention, the appearance of the shape or growth of cells in the process of culture can be observed easily with an optical microscope or the like. In addition, it allows the morphology and function of cells to occur in the same manner as those in vivo. Therefore, the present invention is applicable not only to production of pharmaceuticals and food products, but also to research, development and commercial manufacturing processes in any fields where cell culture including culture of in vivo tissues is needed.

1. A three-dimensional cell culture carrier comprising:
a fibrous structure provided with a three-dimensional space for cell culture, wherein
the fibrous structure is formed of a plurality of interconnected fibers,
each of the fibers is made of a material with a visible light transmittance of 40% or more, when it is formed into a shape having a thickness of 3 mm,
the fiber has an aspect ratio of 1 or more, and
the fiber has a fiber diameter ranging from 100 μm to 700 μm.

2. The three-dimensional cell culture carrier according to claim 1, wherein
the fiber is made of a material with a visible light transmittance of 50% or more, when it is formed into a shape having a thickness of 3 mm.

3. The three-dimensional cell culture carrier according to claim 1, wherein
the fibers have a fiber length ranging from 500 μm to 50000 μm.

4. The three-dimensional cell culture carrier according to claim 1, wherein
the fibrous structure has a porosity ranging from 30% to 70%.

5. The three-dimensional cell culture carrier according to claim 1, wherein
the fibers are glass fibers.

6. The three-dimensional cell culture carrier according to claim 1, wherein
the fibrous structure is composed of sintered glass fibers.

7. The three-dimensional cell culture carrier according to claim 1, wherein
the fibrous structure is divisible into a plurality of sections.

8. A method for cell culture comprising the steps of:
providing a cell culture carrier with cells, and
feeding broth to the cell culture carrier, thereby allowing the cells to grow, wherein
the cell culture carrier is the cell culture carrier of claim 1.
